(19) United States
(12) Patent Application Publication
(10) Pub. No.: US 2013/0123841 A1
(43) Pub. Date: May 16, 2013

(54) Apparatus and Method for a Suture Button

(71) Applicant: Thomas Lyon, Brooklyn, NY (US)

(72) Inventor: Thomas Lyon, Brooklyn, NY (US)

(21) Appl. No.: 13/504,950

(22) Filed: Apr. 28, 2012

Related U.S. Application Data
(63) Continuation of application No. PCT/US09/59054, filed on Sep. 30, 2009.

(51) Int. Cl. A61B 17/04

(52) U.S. Cl. CPC A61B 17/0401
USPC 606/232

(57) ABSTRACT

A suture button apparatus is described that comprises a first button, a plate, a second button and a set of sutures. The first button includes a tubular wall that includes a proximal end portion and an opposed distal end portion that define a central longitudinal axis. An inner surface of the tubular wall defines an aperture aligned with the central longitudinal axis and an outer surface of the tubular wall includes threads. The plate defines at least one aperture that receives and connects with the first button. The set of sutures connect the first button and the second button. The set of sutures connect the first and second buttons.

### BACKGROUND OF THE INVENTION

1. Field of the Invention

The present disclosure relates to the field of surgery and in particular to an apparatus and a method for the stabilization and fixation of bone fractures.

2. Description of the Related Art

Fixation methodologies have been advanced through the use of sutures that interconnect two opposed suture buttons or tissue anchors. These devices facilitate the repair of tissue by using the tension applied by the combination of sutures and buttons to compress the tissue. These devices, however, are limited in their ability to provide stabilization to a fracture.

Treatment for many fractures and ligament tear combinations include a plate and a rigid screw between different bones for stabilization and fixation. As one example, fixation for ankle syndesmosis ligament tears has traditionally included a rigid screw that is positioned to connect a fractured portion of a fibula to the adjacent tibia. This immobilization has certain positive attributes for the healing process, but often requires a second operation to remove the screw. Further, as the patient heals and begins to walk, the screw is subject to several different loads and can be stressed to the breaking point. Certain aspects of this methodology have been improved by the use of suture and button combinations that limit the movement of the fibula relative to the tibia in a flexible construct, but lack the flexibility to address varying stabilization needs for different fractures. These varying needs include overcoming the limited fixation of the suture and button combination in the constrained space requirements of many surgical procedures and the need to position the suture knots such that irritation to the patient is minimized.

In select ankle syndesmosis injuries, such as when a fibula fracture line is in proximity to the ankle joint or when there are several fractures, a surgeon's options are limited by the choice between the traditional rigid screw and current suture button combinations. The problems with the application of suture and button combinations in these injuries include the lack of the ability to separately stabilize the fibula prior to affixing the fibula and tibia together. Additional problems include patient irritation as a result of the flat planar surfaces of the disc shaped button, the layered stacking of the suture button on the surface of the plate and position of the suture knot on the surface of and projecting outward from the suture button towards the skin of the patient.

In another similar application, the options for a surgeon for the fixation of select clavicle fractures that also include tears of the coracoclavicular and coracoacromial ligaments are also limited. The current choices for treatment, rigid screw or suture button combinations, cannot fully provide the advanced capability of stabilizing the clavicle while fixing the stabilized fracture to the coracoid process. Suture based patient irritation remains an issue in this application as the stacking of plate and button positions the suture knot in proximity to the skin of the patient.

A new apparatus and method is needed that can repair tissue through the use of a first threaded button, a second button, a plate with a threaded aperture and a set of sutures. This combination can further improve repairs through the use of a suture button that can be selectively applied in multiple configurations with a plate to stabilize the fracture and affix the position of the fractured bone relative to a nearby bone. The combination can additionally position the knots and terminal ends of the set of sutures within the first button minimizing the depth profile of the integrated plate, suture button and suture knot and thereby reducing or eliminating soft tissue irritation to the patient that is a shortcoming of the prior art.

### SUMMARY OF THE INVENTION

A suture button apparatus is described that comprises a first button, a plate, a second button and a set of sutures. The first button includes a tubular wall that has a proximal end portion and an opposed distal end portion that define a central longitudinal axis. The proximal end portion of the tubular wall includes a proximal terminal end and an external interface adapted to connect to an external drive mechanism for the rotation of the first button. The external drive mechanism includes devices such as a screwdriver. An inner surface of the tubular wall defines an aperture aligned with the central longitudinal axis and an outer surface of the tubular wall includes threads. The plate has a first side and an opposed second side that are connected by a sidewall. The plate defines an aperture that extends through the first side and the second side. The first side receives the first button and the aperture of the plate includes threads that mate with at least a portion of the threads of the first button. The second button includes at least one aperture that receives the set of sutures. The set of sutures connect the first button and the second button. The first button includes a bar that connects to the tubular wall and extends distally into the aperture of the first button. The bar is positioned distal to the proximal terminal end. The inner surface of the tubular wall of the proximal end portion of the first button defines a suture well that receives the terminal end portions of the set of sutures. The suture well is recessed into or distal to the proximal terminal end of the first button. The bar includes a fixation mechanism that fixes the position of the set of sutures relative to the first button. The suture button apparatus has a position for treatment that includes the first button connected to the aperture of the plate and the set of sutures connected to the first and second buttons. The proximal terminal end of the first button is at least flush with the first side of the plate.

The external interface of the proximal end portion is a pair of opposed notches defined in the tubular wall. The notches extend distally from the proximal terminal end of the tubular wall for a predetermined distance to a distal terminal end of the notch. The notches are adapted to receive to the external drive mechanism. The bar is selectively removable from the first button. The fixation mechanism is defined between a sidewall of the removable bar and the inner surface of the tubular wall of the first button. The bar can also include two malleable walls separated by an aperture that receive the set of sutures. The malleable walls are selectively moveable as the fixation mechanism of the bar between an open position and a closed position that fixes the position of the set of sutures between the walls and relative to the first button. The first button includes a first portion that has a first set of threads that interface with the plate and a second portion that has a second set of threads. The first button can also include a shaft and the shaft has threads on the outer surface of the tubular wall.

A method of treating a fractured bone is also described comprising the steps of providing a suture button apparatus that includes a first button, a second button, a set of sutures and a plate. The first button has a tubular wall that includes a distal end and a proximal end that define a through aperture. The first button includes a bar positioned in the aperture and distal to a proximal end. The method includes positioning the second button at a location that supports the fixation of the fractured bone and positioning the first button at least partially through the plate and into an aperture connecting the first and second buttons for the fixation of the fractured bone. The method includes the step of connecting the plate, first button and second button together using the set of sutures. The method can also include the step of threading the first button into a first side of the plate such that the proximal end of the first button is at least flush with a first side of the plate. The method further includes using a fixation mechanism of the bar to fix the position of the set of sutures relative to the first button.

The step of threading can further include threading the first button into the fractured bone. The step of using a fixation mechanism can include fixing the sutures in position by knotting the sutures against the bar. Alternatively, the step of using a fixation mechanism can include fixing the sutures in position using a removable bar that secures the sutures between the bar and an inner surface of a tubular wall of the first button. The step of using the fixation mechanism can also comprise the bar including a pair of malleable walls and the set of sutures extending between the walls. The fixation mechanism including crimping the malleable walls together fix the position of the set of sutures relative to the malleable walls.

### BRIEF DESCRIPTION OF THE DRAWINGS

The foregoing and other features of the present invention will become more apparent upon consideration of the following description taken in connection with the accompanying drawings wherein:

FIG. 1 is a perspective view of a suture button apparatus constructed in accordance with the present disclosure that includes a first button with a fixed bar;

FIG. 2 is a perspective view of the suture button apparatus of FIG. 1 with the first button further including a shaft;

FIG. 3 is a cross-sectional front and top perspective view of the first button of the suture button apparatus of FIG. 2 taken along lines 3-3;

FIG. 4 is a side and top perspective view of a plate of the suture button apparatus of FIG. 2;

FIG. 5 is a side and top perspective view of a second button of the suture button apparatus of FIG. 2;

FIG. 6 is a side and top perspective view of the first button of FIG. 2 in a first position that further includes a removable bar;

FIG. 7 is a side and top perspective view of the first button of FIG. 6 in a second position that includes the first button and the bar connected together;

FIG. 8 is a side and top perspective cross-sectional close up view of the first button of FIG. 7 taken along lines 8-8 showing the first button and bar connected together;

FIG. 9 is a side and top perspective view of the first button with a malleable bar in a first position;

FIG. 10 is a side and top perspective cross-sectional close-up view of the first button with the malleable bar of FIG. 9 taken along lines 10-10, the first button further including sutures in the aperture defined between the malleable wall sections of the bar;

FIG. 11 is a side and top perspective view of the first button of FIG. 9 with the malleable bar in the second position fixing the position of the set of sutures;

FIG. 12 is a side and top perspective cross-sectional close-up side view of the first button with malleable walls of FIG. 11 taken along lines 12-12;

FIG. 13 is an idealized side cross-sectional view of a first step of an operative employment process of the suture button apparatus of FIG. 2 for the fixation of ankle syndesmosis;

FIG. 14 is an idealized side cross-sectional view of a second step of the operative employment process of the suture button apparatus of FIG. 13;

FIG. 15 is an idealized side cross-sectional close-up view of the second step of the operative employment process showing the first button of the suture button apparatus of FIG. 14;

FIG. 16 is an idealized side cross-sectional close-up view of the second step of the operative employment process of the suture button apparatus of FIG. 6 for the fixation of ankle syndesmosis;

FIG. 17 is an idealized side cross-sectional close-up view of the second step of the operative employment of the suture button apparatus of FIG. 9 for the fixation of ankle syndesmosis;

FIG. 18 is an idealized side view of a fractured clavicle that is at least partially torn from the coracoclavicular and coracoacromial ligaments;

FIG. 19 is an idealized side view of the operative employment of the suture button apparatus of FIG. 9 aligned for treating the clavicle and torn ligaments of FIG. 18; and

FIG. 20 is an idealized side view of the suture button apparatus of FIG. 19 in a second position.

### DETAILED DESCRIPTION OF THE INVENTION

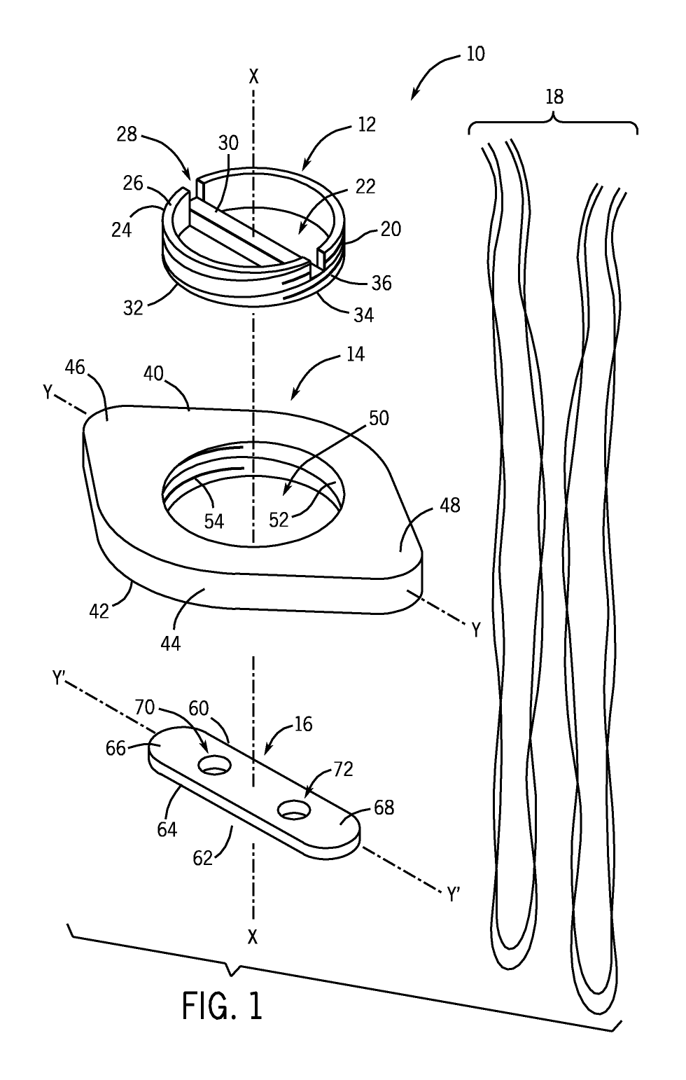

Referring to FIG. 1, the present disclosure is directed to a suture button apparatus 10 that comprises a first button 12, a plate 14, a second button 16 and a set of sutures 18. First button 12 and second button 14 are connected by set of sutures 18. First button 12 connects to plate 14.

First button 12 has an annular tubular wall 20 that defines a centrally aligned axis-X. An inner surface of the tubular wall defines an aperture 22 aligned with axis-X. Tubular wall 20 has a proximal end portion 24 and a proximal terminal end 26. Proximal end portion 24 includes a pair of notches 28 that extend distally from terminal end 26. First button 12 includes a bar 30 that connects to an inner surface of tubular wall 20. Tubular wall 20 has a distal end portion 32 and a distal terminal end 34. The outer surface of tubular wall 20 defines threads 36.

Notches 28 are longitudinally aligned channels in tubular wall 20. In this preferred embodiment, each notch 28 defines sidewalls in tubular wall 20 that are approximately parallel and aligned with the opposing notch 28. Notches 28 extend from rim 26 for a predetermined distance to a distal terminal end. Notches 28 can be through holes in tubular wall 20 that interrupt the continuity of proximal terminal end or rim 26 or grooves defined in tubular wall 20 that only partially interrupt the continuity of rim 26. In this preferred embodiment of notches 28, the channels and sidewalls are diametrically opposed and aligned.

Bar 30 includes a fixation mechanism for set of sutures 18. The fixation mechanism of bar 30 as defined herein facilitates the fixing of sutures 18 and/or fixes the position of sutures 18 relative to first button 12. Bar 30 is positioned distal and in proximity to rim 26 such the fixation mechanism accommodates the retention of the proximal terminal end portions of sutures 18 below rim 26 of first button 12.

Bar 30 has a beam configuration that is connected to tubular wall 20 in aperture 22 distal to proximal rim 26. Bar 30 is preferably connected to tubular wall 20 in proximity to the distal terminal end notches 28. Bar 30 in one preferred embodiment is a rigid structure fixedly connected at both opposing terminal ends to tubular wall 20. It is understood however, that bar 30 can take broad range of structural configurations, connect to tubular wall 20 by differing mechanical methods to include removably positionable and connect at any angular orientation relative to the longitudinal axis-X and tubular wall 20. Structural configurations can include a rigid beam, cantilever beam, bifurcated structure, malleable structure and resilient structure each of which can provide advantages for the fixation mechanism. Bar 30 can have any cross-sectional shape that provides the required structural support to include, for example, a flat planar structure, circular cross-section, and/or an arcuate or angled shape.

Bar 30 extends a predetermined distance in the distal direction of the longitudinal axis-X from proximal end portion 24. The distance of bar 30 in the direction of the longitudinal axis can vary depending upon the intended application of apparatus 10 and can include extending beyond distal rim 34.

Plate 14 includes a first or proximal side 40 and an opposed second or distal side 42 that are connected by a sidewall 44. Plate 16 has a planar disc shape that includes a first end portion 46 and an opposed second end portion 48. An axis-Y is aligned with first end portion 46 and second end portion 48. Plate 16 preferably has an elongate shape and axis-Y is aligned with an elongate axis defined between first end portion 46 and second end portion 48.

Plate 14 in this preferred embodiment can provide functions such as increasing the surface area interface of first button 12, contribute to the stabilizing of an area and distribute the compression load that is applied by suture button apparatus 10 across a larger area. The structural shape and area of interface defined by distal side 42 of plate 16 can vary with the intended application.

An aperture 50 is defined by an interior wall 52 of plate 14 that is aligned with axis-X and perpendicular to axis-Y. Interior wall 52 also defines threads 54 that mate with threads 36 to position proximal rim 26 of first button 12 at the desired connected position in aperture 50 relative to first side 40 of plate 14. The connected position of first button 12 in plate 14 includes proximal end portion 24 and/or proximal end 26 at least flush with first side 40. At least flush as defined herein is level with side 40 of plate 14 and/or distal to side 40.

The first button 12 and plate 14 interface can further include a locking mechanism to securely connect button 12 in the connected position. For example, threads 54 can be constructed to lock first button 12 at the connected position such that rim 26 is at least flush with and/or distal to first side 40 of plate 14. Additional features that can be provided in the connected position include torque restraints for the engagement/disengagement of the locking mechanism, a ratcheting function and/or the free rotational floating of button 12 in position in plate 12.

Second button 16 has an elongate narrow approximately planar shape that includes a first side 60 and an opposed second side 62 connected by an edge or side 64. Button 16 has a first end portion 66 and an opposed second portion 68 that define a longitudinal axis-Y′. At least two apertures 70 and 72 are defined button 16 that are approximately aligned with axis-X and approximately perpendicular to axes Y′. Alternatively, button 16 can define a single aperture that is at least partially divided by a bar 30 and selectively includes threads on at least a portion of side 64 that engage with threads defined in an aperture in a second plate.

Set of sutures 18 preferably includes four sutures employed with fixation apparatus 10. In one preferred embodiment, a first pair of sutures of set of sutures 18 can be fabricated of any material and strength rating. A second pair of sutures of suture of set of sutures 18 can also be fabricated of a broad range of materials to include metal wire, but is suitable for fixation applications and preferably with a strength rating of zero (0). The number of sutures in set of sutures 18 can vary depending upon the application of suture button apparatus 10.

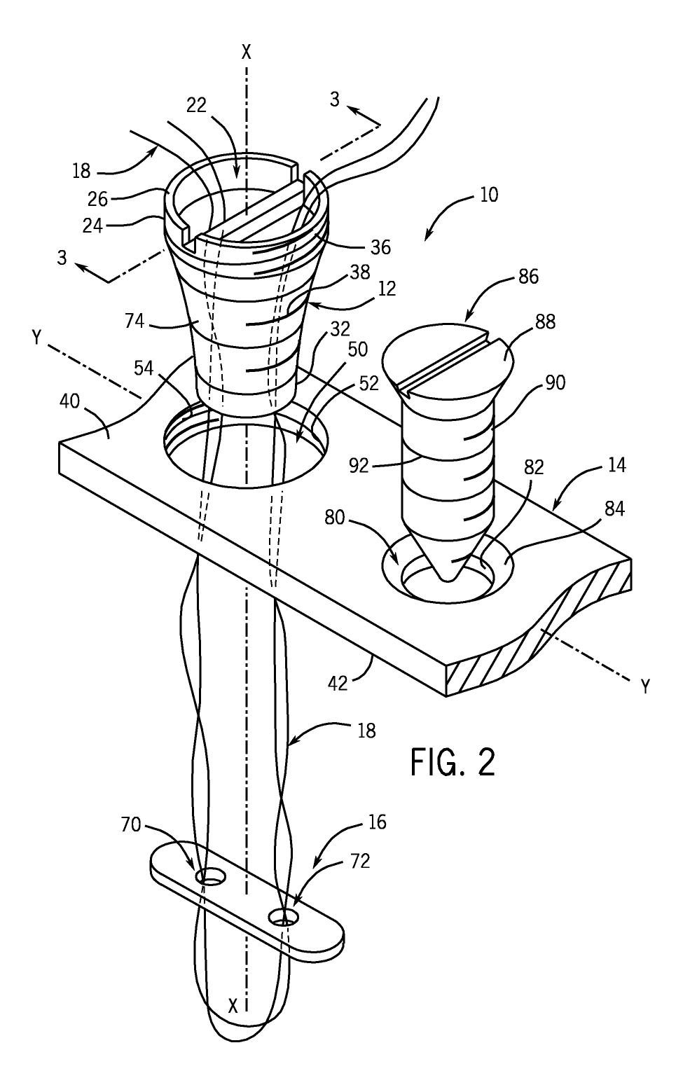

As shown in FIG. 2, first button 12 has a first portion of tubular wall 20 and can further include a second portion of tubular wall 20 that includes a distally extending shaft 74. As described previously, wall 52 of plate 14 defines aperture 50 and threads 54 that mate with threads 36 on proximal end portion 24. Set of sutures 18 are shown threaded through aperture 22 of first button 12, plate 14, through apertures 70 and 72 of second button 16 and then again through aperture 22.

The outer surface of shaft 74 preferably includes threads 38. Shaft 74 can also have alternative outer surfaces to include a smooth wall, protuberances aligned with the longitudinal axis-X as well as grooves. Shaft 74 can have a uniform diameter or a distally directed decreasing taper depending upon the intended application.

Plate 14 has an extended length along the longitudinal axis-Y in this preferred embodiment and defines a plurality of apertures between proximal side 40 and distal side 42. The plurality of apertures includes at least one aperture 50 and preferably includes at least one aperture 80. Annular wall 52 in plate 14 defines aperture 50 and threads 54. Tubular wall 82 and a countersink 84 define aperture 80. Flat head type fasteners 86 are adapted to connect in a close fitting relation with countersink 84. Fasteners 86 and button 12 have a first position displaced from plate 14 and a second position connected to plate 14 such the proximally directed surface of head 88 and proximal rim 26 are at a flush or recessed position relative to first side 40 of plate 14.

First button 12, plate 14 and second button 16 can be of any material suitable for medical applications and functions defined herein to include metals, composites and polymer materials. This can include disposable, reusable and bioabsorbable materials.

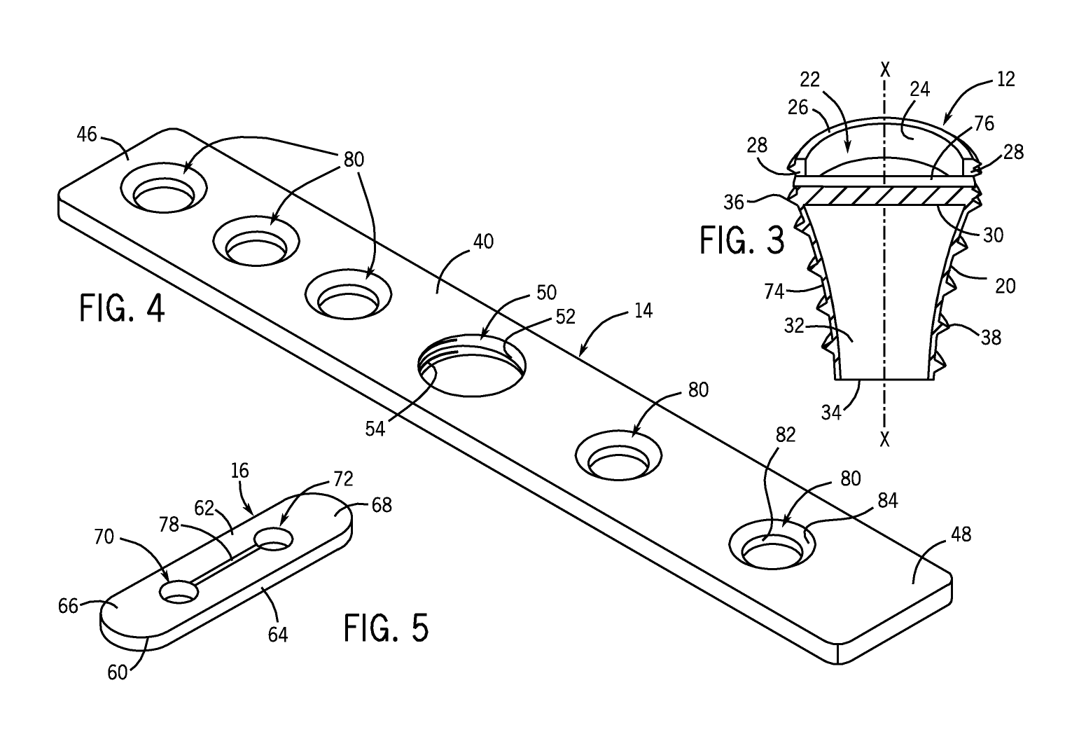

Referring now to FIGS. 2 and 3 a proximal side 76 of bar 30 is aligned with the distal terminal end of notches 28 in tubular wall 20. Side 76 can include a flat planar surface that defines a channel, for example, that receives the distal tip of the external drive mechanism. Tubular wall 20 can further include fillets or flanges that reinforce bar 30 and/or the connection between bar 30 and wall 20.

The diameter of shaft 74 of first button 12 has a distally directed decreasing arcuate taper from in proximity to proximal end portion 24 to distal end 34. Threads 38 can be distinct from threads 36 and include alternative constructions such as for engaging bone. Terminal end 34 of distal end portion 32 preferably has a rounded non-cutting edge 34. The length of shaft 74 can vary depending upon the intended application, but extends along axis-X distal to plate 14 in the connected position of first button 12 and plate 14.

As shown in FIG. 4, plate 14 is an elongate bar that includes six apertures in this one exemplary embodiment between first longitudinal end 46 and second longitudinal end 48. The six apertures include five apertures 80 and one aperture 50. Tubular wall 52 of aperture 50 defines threads 54 that mate with the threads of 36 of first button 12. Plate 14 can be a rigid or a flexible member that provides a controlled degree of stiffness for a given application. Similarly, the number and type of apertures in plate 14 can vary depending upon the intended application.

Referring to FIG. 5, distally directed second side 62 of second button 16 includes a slot 78 that extends between apertures 70 and 72. Edge 64 in proximity to first end portion 66 and second end portion 68 includes a rounded taper. Second button 16 can also have a structure similar to that of first button 12 in which a single aperture is at least partially divided by bar 30.

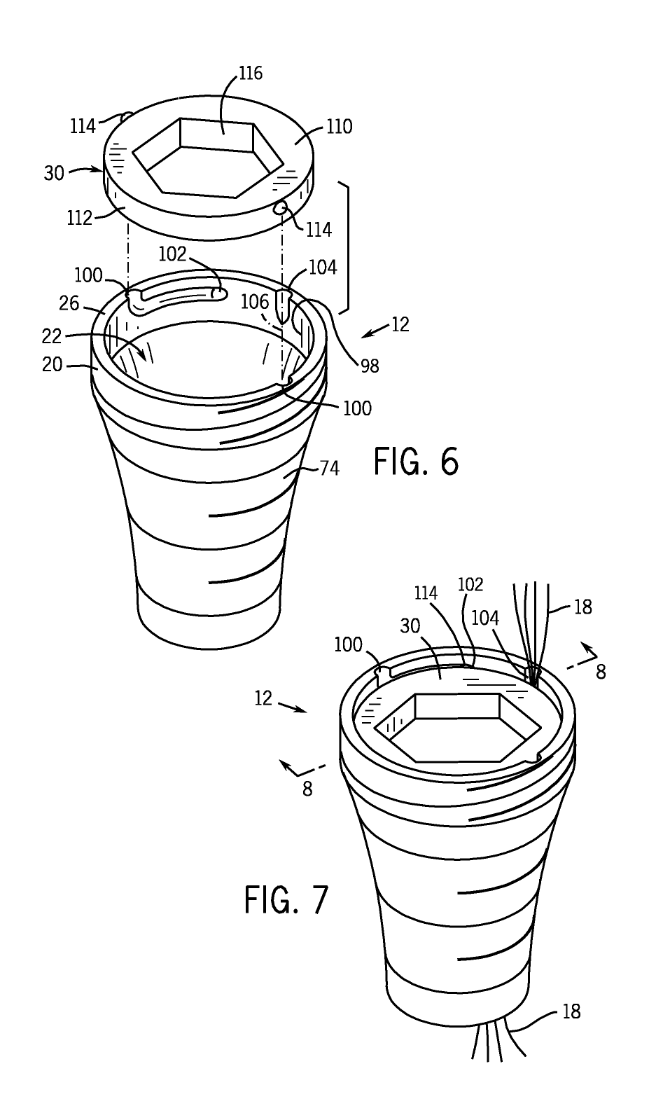

As shown in FIG. 6, the fixation mechanism of first button 12 includes an annular bar 30 that interfaces with an inner surface 98 of tubular wall 20. A set of channels 100 are defined in tubular wall 20 inner surface 98 that extend between rim 26 and a predetermined distance in inner surface 98. Set of channels 100 includes one or more channels 100 defined in the inner surface 98 of wall 20. Each channel 100 has a distal terminal end 102 that can further include a locking mechanism. Channels 100 are limited in length for ease of connection and preferably extend approximately ninety (90) degrees or less along inner surface 98 between rim 26 and terminal end 102.

Inner surface 98 preferably defines two diametrically opposed channels 100. The initial portion of each channel 100 in proximity to rim 26 is approximately aligned with the longitudinal axis similar to notch 28 (see FIG. 3). The angle of the remaining portion of each channel 100 relative to the longitudinal axis can vary to include for example helical, angled or perpendicular depending upon the intended application and desired position of bar 30 relative to rim 26.

First button 12 also includes a notch 104 on inner surface 98 of tubular wall 20. Notch 104 extends a predetermined radial depth into tubular wall 20 and a predetermined distance distally approximately aligned with the longitudinal axis from rim 26. Notch 104 preferably terminates prior to the distal end of proximal end portion 24 such that there is an extended portion 106 of inner surface 98 of tubular wall 20. Extended portion 106 is preferably longitudinally aligned with notch 104, at least the full inside diameter of the inner surface 98 and can extend distally along inner surface 98 into shaft 74. Notch 104 and/or the aligned extended portion 106 can selectively include mechanical enhancements that can include for example knurling, undulations, capturing recesses that use a slot or flap, or chemical, such as an adhesive suitable for surgical applications. Notch 104 and extended portion 106 are preferably positioned independent of channel 100, but distal terminal end 102 can, for example, include notch 104.

Bar 30 in this preferred embodiment has an annular shape that includes a first side 110 and an opposing side (not shown) that are connected by a sidewall 112. A close fitting relation is defined between sidewall 112 and extended portion 106. Sidewall 112 includes a set of protuberances 114 that correspondingly interface with set of channels 100 for the rotation and securing of bar 30 with tubular wall 20.

The fixation mechanism in this preferred embodiment of first button 12 includes the close fitting relation between sidewall 112 and extended portion 106 on inner surface 98 of tubular wall 20. Bar 30 is movable between a first position disconnected from button 12 to a connected position with button 12 in which set of protuberances 114 of bar 30 are positioned in terminal ends 102 of set of channels 100 of button 12.

Bar 30 also defines an external interface 116 for rotation by the external drive mechanism. External interface 116 is shown in this embodiment as defining a hexagonal shaped aperture that is in communication with aperture 22 of tubular wall 20, but it is understood that bar 30 can be a solid disc that define any shape of aperture for external interface 116. Equivalents of external interfaces 116 include a slot, a crossbar, square, Phillips and star type interfaces commonly employed as turning interfaces for fasteners. As shown in this exemplary embodiment, the external drive mechanism includes a hex drive interface for external interface 116.

As shown in FIGS. 6 and 7, bar 30 is connected to first button 12 by aligning and inserting set of protuberances 114 into set of channels 100 at rim 26. Bar 30 is then rotated to position and securely connect set of protuberances 114 in terminal ends 102 of each channel 100 to define the connected or second position of bar 30. Extended portion 106 and sidewall 112 are secured in a close fitting relation such that set of sutures 18 are fixed in position relative to first button 12.

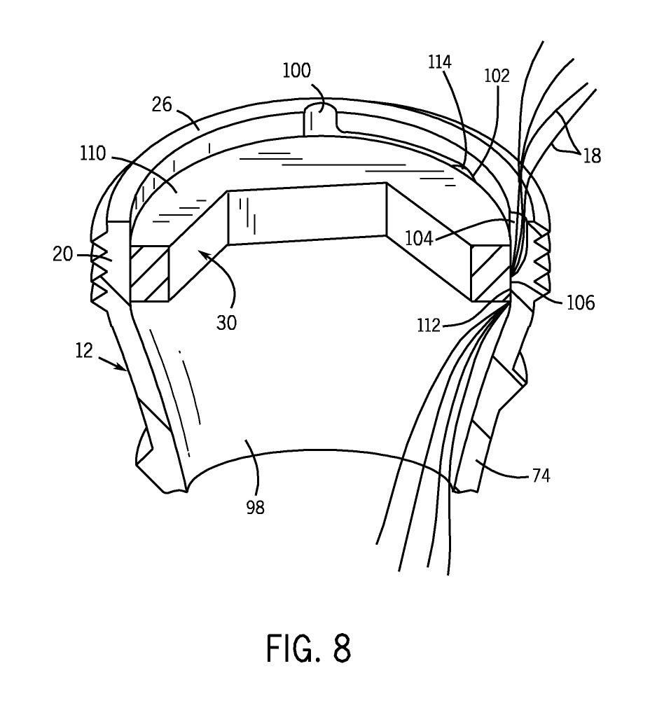

Referring now to FIG. 8, the interface that defines the fixation mechanism can extend distally a predetermined distance to include an increased length of sidewall 112 and an increased length of extended portion 106 along inner surface 98 of shaft 74. The extended length of interface can be varied to achieve the desired increase in the applied forces for the fixation of set of sutures 18. First side 110 of bar 30 is at least flush with or distal to rim 26 in the second position such that set of sutures 18 can be readily accessed through notch 104 for cutting or knotting through external interface 116, for example, after being fixed in position.

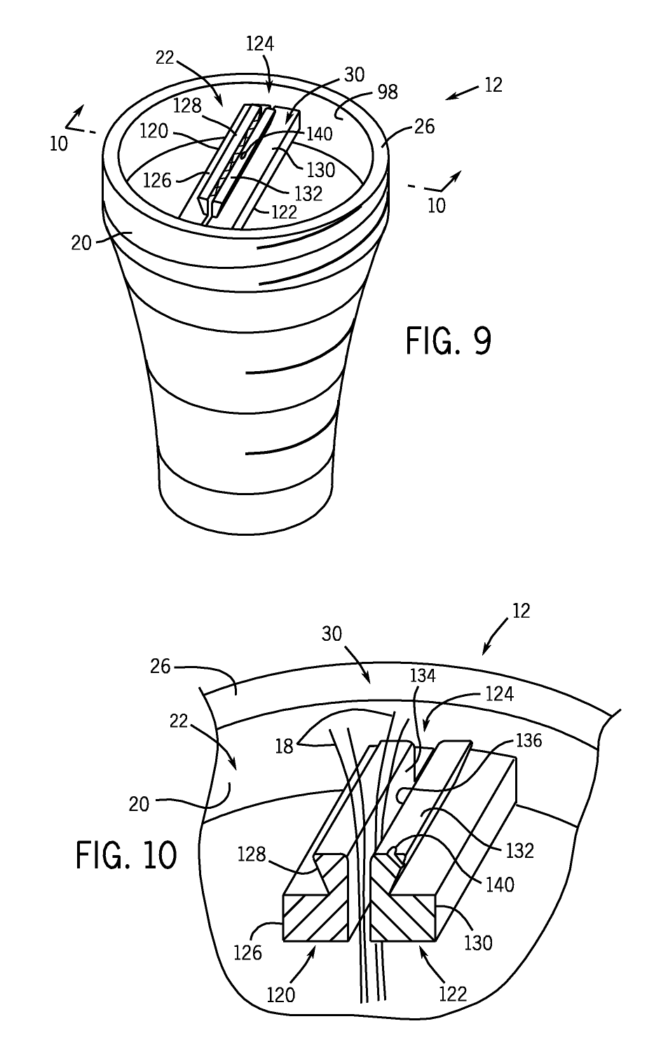

Referring now to FIG. 9, bar 30 can also include a first portion 120 and a second portion 122. An aperture 124 is defined in a first position of bar 30 between portions 120 and 122 that is in fluid communication with aperture 22 and suitable for the passage of set of sutures 18. At least a part of first portion 120 and second portion 122 are malleable such that they can be deformed to provide a joint that securely fixes and sets the position of sutures 18. Bar 30 is positioned distal to proximal rim 26.

As shown in FIGS. 9 and 10, first portion 120 has a base 126 and a wall section 128 and second portion 122 has a base 130 and a wall section 132. Base sections 126 and 130 connect to tubular wall 20 of first button 12. Wall sections 128 and 132 connect to base sections 126 and 130, respectively and are separately moveable such that each wall section 128, 132 can be repositioned relative to their respective bases 126 and 130 to fix the position of set of sutures 18 relative to first button 12. Wall sections 128 and 132 are preferably fabricated of an at least partially malleable material that is suitable for providing a secure connection between wall sections 128 and 132 and set of sutures 18. Wall sections 128 and 132 can include undulations, apertures and/or folds to facilitate the displacement of walls 128 and 132 relative to bases 126 and 132.

The connection between wall sections 128 and 132 and their respective bases 126 and 130 is preferably such that once wall sections 128 and 132 are displaced from their initial open position to a second position, bases 126 and 130 continue to structurally support the moved wall sections 128 and 132. The movement of wall sections 128 and 132 can be angular and/or translational and include portions of bases 126 and 130.

In addition, wall sections 128 and 132 have opposing faces 134 and 136 that are shown as planar, but can also can include mechanical connection enhancements such as undulations, teeth, knurling, etc. as well as include chemical adhesives for bonding that can provide an enhanced connection. Aperture 124, defined between portions 120 and 122, is in fluid communication with aperture 22 of button 12. Wall sections 128 and 132 have opposing terminal ends in proximity to tubular wall 20 that can be flared or alternatively overlapping depending upon the intended application of button apparatus 10.

Wall section 132 includes a secondary fixation mechanism. In this preferred embodiment, an aperture 140, that is a through hole that extends between the proximally directed surface of wall 132 and the opposing side of the face 136, provides the secondary capability to securely fix the terminal ends of sutures 18 using known means such as a surgical knot after being fixed in position by faces 128 and 132. The secondary fixation mechanism provides a layer of redundancy. Aperture 140 can be a hole, slot or recessed cleat and can have a matching counterpart on wall 128. It is understood that aperture 140 can be positioned in any portion of bar 30 to include base portions 126 and 130. Additional secondary fixation mechanisms can include bases 126 and 130 as well as mirror wall sections connected to the distal side of bases 126 and 130.

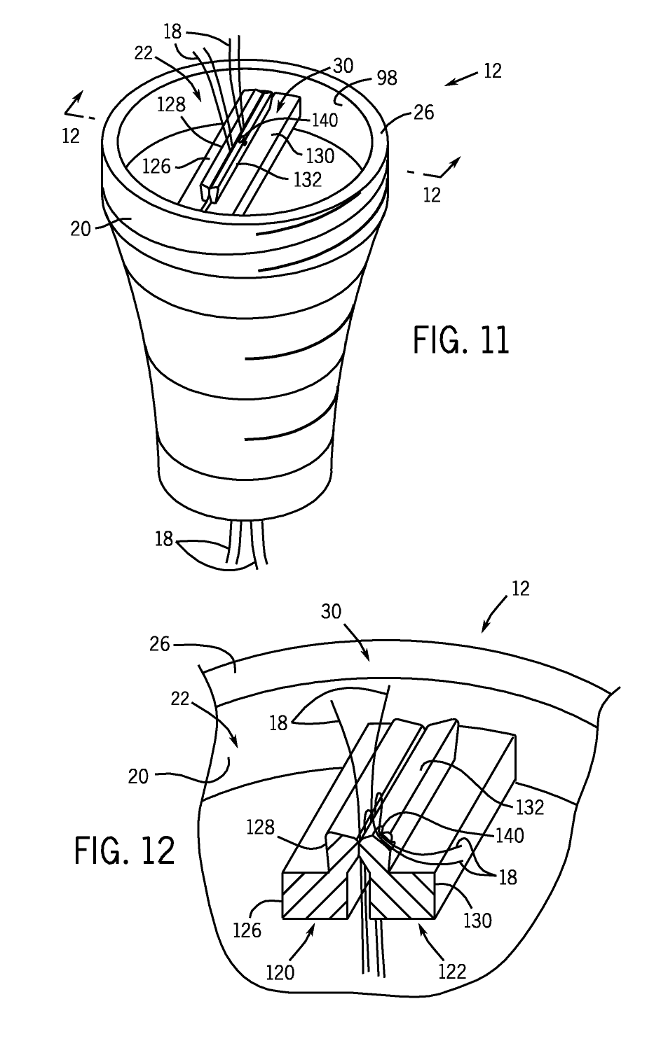

Referring now to FIG. 11, walls 128 and 132 are moved to a second closed position are compressed together to fix set of sutures 18 relative to first button 12. Bases 126 and 132 remain in position connected to tubular wall 20. Opposing faces 134 and 136 provide a secure mechanism to fix sutures 18 and the secondary fixation mechanism can be optionally included and/or employed.

As shown in FIG. 12, bar 30 wall portions 128 and 132 are crimped together into a close fitting relation that securely fixes sutures 18 in position relative to first button 12. Bases 126 and 130 connect to tubular wall 20 and support the movement of walls 128 and 132. Bar 30 is positioned distal to rim 26. The terminal ends of two individual sutures 18 are extending from bar 30 and the opposing two terminals of the sutures 18 are threaded through aperture 140 to provide a secondary fixation mechanism. Alternatively, the secondary fixation mechanism can be a fixed central wall that subdivides malleable wall portions 128 and 132 into two set of approximately parallel wall portions 128 and 132 and sutures 18 are tied across the central wall.

As shown in FIGS. 2 and 13, first button 12, plate 14, second button 16 and set of sutures 18 of suture button apparatus 10 are in one exemplary operative employment for the treatment of ankle syndesmosis. A hole is drilled through the tibia 1 and fibula 2 that is preferably in proximity to and below the fracture 4 in fibula 2. Aperture 50 of plate 14 is positioned over the hole and in close proximity to the fibula 2.

One or more sutures of set of sutures 18 is connected to a penetrating device 5 and second button 16 using apertures 70 and 72. Penetrating device 5 or a similar instrument such as a probe, needle or a wire is used to pass second button 16 through aperture 50, the hole drilled in fibula 2 and tibia 1. Second button 16 is positioned on the opposing side of the hole and repositioned over the medial opening of tibia 1 to be approximately flat against the tibia and approximately aligned with plate 14. Set of sutures 18 connect second button 16 to first button 12 through plate 14. Each suture 18 is run into and the terminal ends separated in first button 12 such that the terminal ends of each suture 18 is on the opposing sides of bar 30.

In this exemplary operational employment, plate 14 has one aperture 50 and five apertures 80 as described previously. Fasteners 86 and/or first button 12 are prepared for connection through plate 14 to fibula 2. Set of sutures 18 connect button 12, plate 14 and second button 16. The connection is preferably made using a set of sutures 18 that initially includes four sutures in set of sutures 18. It is understood that second button 16 can also take the configuration of a plate 14 and first button 12 combination as described herein.

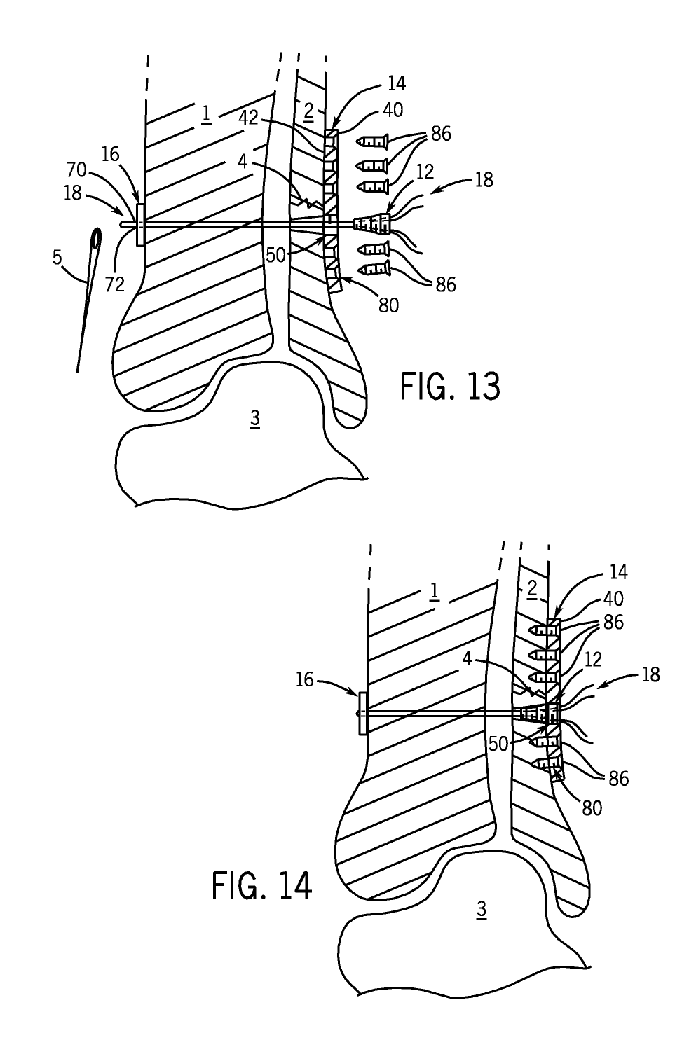

Referring now to FIGS. 2 and 14, fasteners 86 are inserted into first side 40 of plate 14 and screwed into fibula 2. The heads 88 of fasteners 86 in the connected position are countersunk into countersink 84 of plate 14 such that the proximally directed surfaces of heads 88 are at least flush with first side 40. First button 12 is inserted into aperture 50 on first side 40 of plate 14 and the aperture in fibula 2. The external drive mechanism is employed to connect button 12 and plate 14. In this example, the drive mechanism is a standard screwdriver that is positioned in notches 28 in proximal end portion 24 and rotated to engage threads 38 of shaft 74 with fibula 2 and threads 36 with threads 54 of aperture 50. Button 12 and plate 14 are preferably placed in the connected position in approximately one-quarter turn or less. Once in the connected position, an optional locking mechanism can secure first button 12 in position. Specific additional functions of button 12 in the connected position include being captured and rotating freely as well as defining a torque threshold wherein the engagement of the locking mechanism requires a first force and the extraction of button 12 from plate 14 requires a second force that is greater than a first force.

The depth of threads 54 of plate 14 and threads 36 enables the positioning of proximal end portion 24 of first button 12 in the connected position flush or distal to first side 40 of plate 14. Proximal end portion 24 can optionally extend past second side 42 of plate 14 and engage fibula 2.

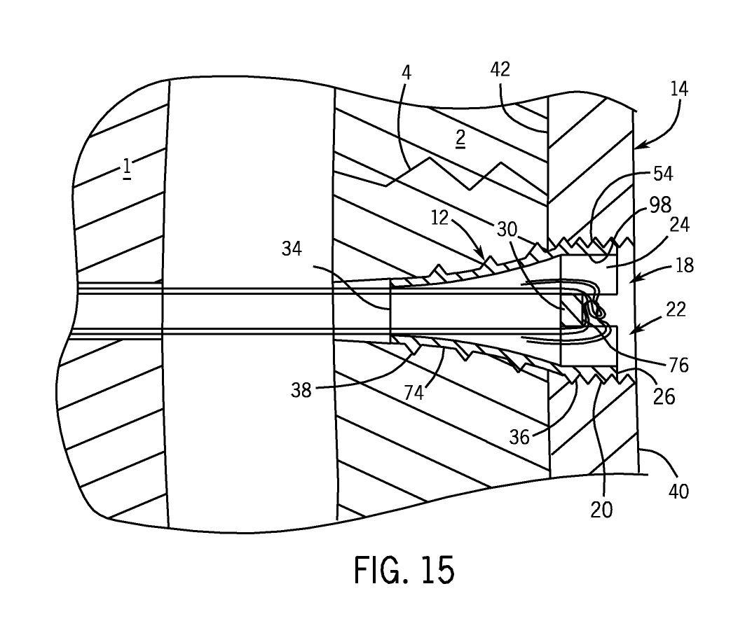

As shown in FIGS. 14 and 15, set of sutures 18 are drawn taught between second button 16 and first button 12 in plate 14. The connection of sutures 18 with bar 30 of first button 12 places suture button apparatus 10 in the second position stabilizing fibula 2 and fixing the relative position of fibula 2 using second button 16 positioned on the opposing side of the tibia 1 medial hole. The optional shaft 74 and threads 38 extend into fibula 2 for a predetermined distance and provide an additional element of stabilization of fracture 4 of fibula 2. The additional function of a ratchet type rotating engagement of threads 36 and 54 of first button 12 and plate 14 can be selectively employed to adjust the tension on fixed set of sutures 18.

The inner surface 98 of tubular wall 20 of proximal end portion 24 and bar 30 define a suture well in the proximal portion of aperture 22. The terminal ends of set of sutures 18 are retained distal to rim 26 and/or first surface 40 of plate 14. The positioning of the knots and terminal ends of the set of sutures 18 in the well and distal to rim 26 and/or first side 40 eliminates the irritation and potential infection that can be encountered by patients when the knots and terminal ends of the set of sutures extend proximal to plate 14.

In addition, as required, bar 30 can include an arcuate shape that extends distally into proximal end portion 24 to accommodate an additional volume in the suture well defined by proximal end portion 24. Similarly a resilient bar 30 would place set of sutures 18 under a predetermined amount of bias. The selective inclusion of a channel in bar 30 and fillets in tubular wall 20 can further aid the connection to and rotation of button 12 by the drive mechanism, such as a bifurcated screwdriver 144 (See FIG. 19).

Once the treatment period is completed, suture button apparatus 10 can be released by cutting set of sutures 18. First button 12, fasteners 86 and plate 13 can be removed. Second button 16 can be withdrawn through the hole drilled through the tibia 1 and fibula 2 or alternatively removed from the medial side of the opening and as required any sutures 18 withdrawn.

As shown in FIGS. 1 and 14, suture button apparatus 10 is selectively employable as a disc with an abbreviated length along axis-X and plate 14 with an abbreviated length along axis-Y that is appropriate for select confined operational uses, for example. The combination of button 12 and plate 14 accommodates the knotting and retention of set of sutures 18 in the suture well and provides the less rigid fixation in proximity to fracture 4 using set of sutures 18 through tibia 1 and fibula 2 in the second position. Plate 14 provides additional load bearing function relative to the patient and can be extended to provide additional points of fixation.

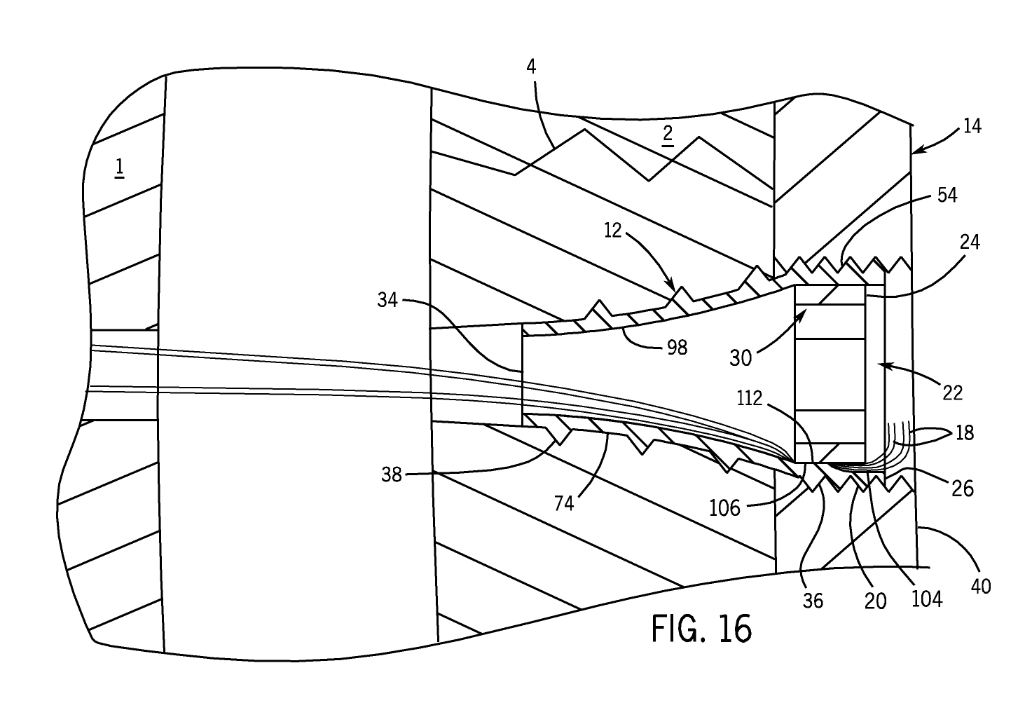

Referring now to FIGS. 2, 6-8 and 16, first button 12, plate 14, second button 16 and set of sutures 18 of suture button apparatus 10 are in one exemplary operative employment for the treatment of ankle syndesmosis. A hole is drilled through the tibia 1 and fibula 2 that is preferably in proximity to and below the fracture 4 in fibula 2 and suture button apparatus 10 is connected together such that the set of sutures 18 extend through aperture 22 as described previously. Button 12 is threaded into plate 14 using the drive mechanism to interface with portions of channel 100 in proximity to rim 26 that are aligned with the longitudinal axis and similar to notches 28. First side 110 of bar 30 is in the connected position and flush or distal to first side 40 of plate 14. Set of sutures 18 extend through aperture 22 to second button 16. Button 12 is inserted at least partially into aperture 80 of plate 14 and fibula 2. Threads 36 are at least aligned for engagement with threads 54 of aperture 80 and the drive mechanism is positioned in the longitudinally aligned portions of each channel 100 and rotated about the longitudinal axis. Button 12 is rotated into the connected position flush with or distal to side 40 of plate 14.

Sutures 18 are pulled taught and are preferably positioned along extended portion 106 of inner surface 98 and in notch 104. Bar 30 is positioned in aperture 22 of first button 12 and protuberances 114 engage channels 100. The drive mechanism, such as a hex drive, connects to external interface 116 and rotates bar 30 to engage channels 100 with protuberances 114 and side 112 of bar 30 with extended portion 106 of tubular wall 20. Sutures 18 are fixed in position between inner surface 98 of tubular wall 20 along extended portion 106 and side 112. Additional secondary fixation can include knotting sutures 18 in notch 104 and between side 112 and inner surface 98 of tubular wall 20.

Once the treatment period is completed, set of sutures 18, are released by using the drive mechanism to rotate bar 30 in the opposite direction from its initial installation. If set of sutures 18 are knotted then the knots and/or sutures can be cut and the knots removed. First button 12 and fasteners 86 are withdrawn from the fibula 2 and plate 14 removed. Second button 16 can be withdrawn as described previously.

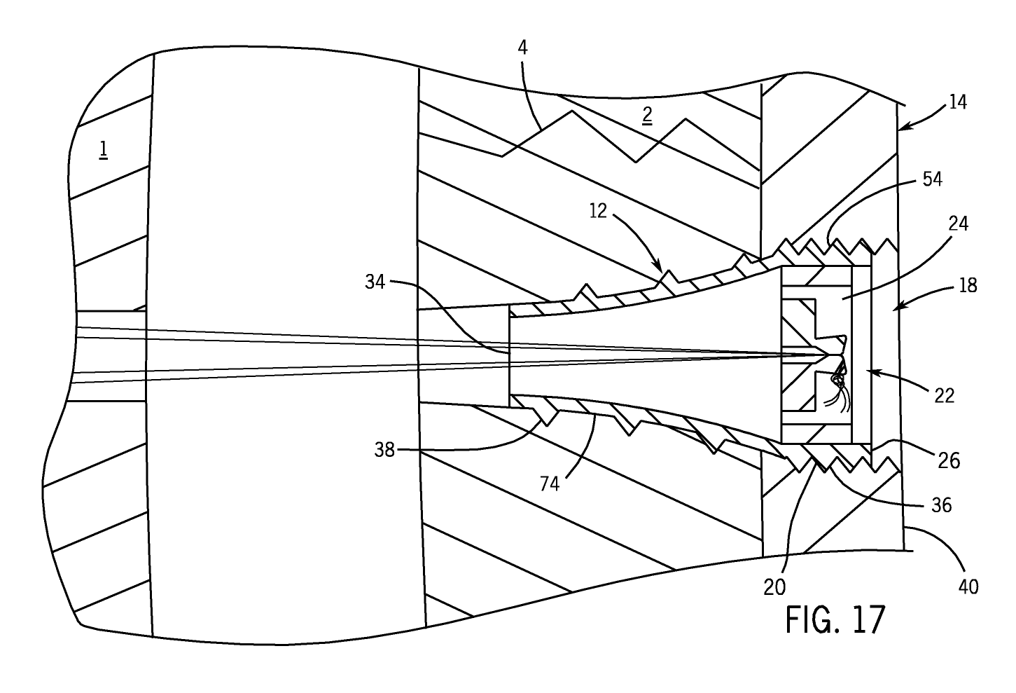

Referring now to FIGS. 9-12, 14, 15 and 17, first button 12, plate 14, second button 16 and set of sutures 18 of suture button apparatus 10 are in another exemplary operative employment for the treatment of ankle syndesmosis. As described previously, a hole is drilled through the tibia 1 and fibula 2 and suture button apparatus 10 is connected together such that the set of sutures 18 extend through apertures 22 and 124. Fasteners 86 are inserted into plate 14 and fibula 2. First button 12 is inserted into plate 14 and into fibula 2. The drive mechanism, such as bifurcated device 146 (See FIG. 19), rotates and engages first button threads 36 with threads 54 of plate 14 to place button 12 in the connected position such that rim 26 is at least flush with first side 40. Alternatively, the drive mechanism is a screwdriver that is positioned in aperture 124 and rotated against bases 126 and 130 to rotate first button 12 into the connected position.

Set of sutures 18 are pulled taught and a tool, such as a crimper or pair of pliers 146 (See FIG. 19) is used to press at least walls 128 and 132 together as the fixation mechanism of bar 30 to fix set of sutures 18 relative to first button 12. Secondary fixation can be accomplished, as desired, using aperture 140 to separate the opposing ends of sutures 18 and knot sutures 18 over wall 132 and within the suture well.

Once the treatment period is completed, set of sutures 18 are released by using a pair of pliers or crimpers 146 (See FIG. 19) to compress malleable walls 128 and 132 at their opposing ends in proximity to tubular wall 20 and/or by cutting set of sutures 18 distal to bases 126 and 130. First button 12, second button 16 set of sutures 18, fasteners 86 and plate 14 can then be removed as described previously.

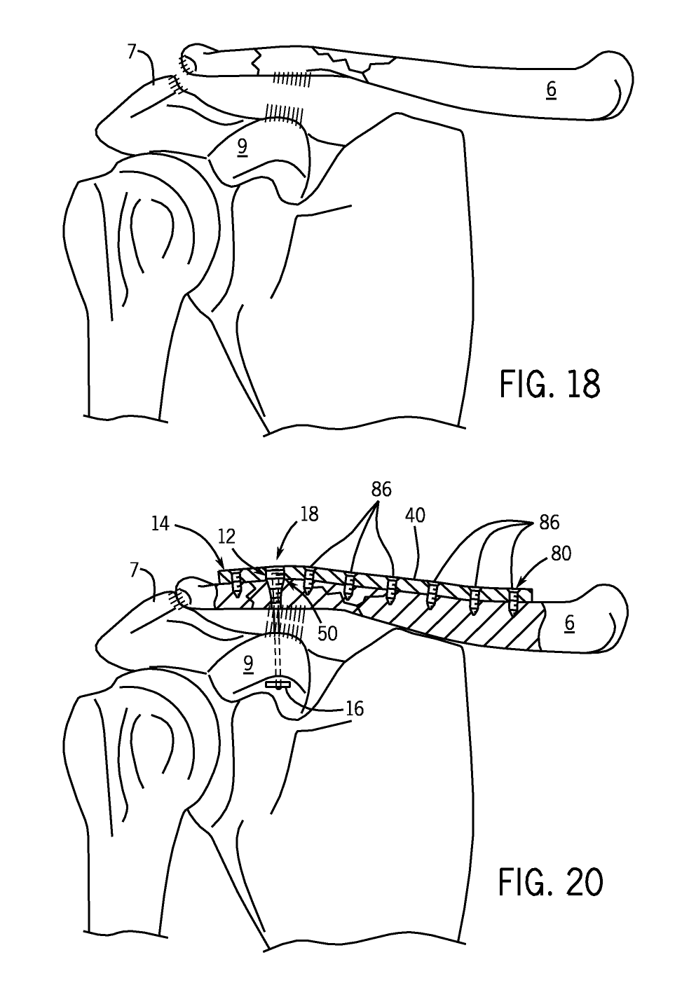

As shown in FIGS. 18 and 19, another exemplary fracture that suture button apparatus 10 can be operatively employed is a clavicle 6. In this instance, clavicle 6 is fractured and the coracoclavicular and coracoacromial ligaments are at least partially torn detaching clavicle 6 from an acromion 7 and a coracoid process 9. A hole is drilled through the clavicle 6 and coracoid process 9 that is preferably in proximity to one of the fractures in clavicle 6. In this exemplary operational employment plate 14 has one aperture 50 and five apertures 80 as described previously. Aperture 50 of plate 14 is aligned with the hole in clavicle 6. Fasteners 86 and first button 12 are aligned with their respective apertures 80 and 50.

Second button 16 is connected to sutures 18 and positioned on the opposing side of the hole in coracoid process 9 and repositioned over the opening as previously described for ankle syndesmosis. Set of sutures 18 connect second button 16 to first button 12 through plate 14. Sutures 18 are run through aperture 22 and 124 and separated into opposing terminal ends on the proximal side of bar 30.

Fasteners 86 and/or first button 12 are prepared for connection through plate 14 to clavicle 6. Set of sutures 18 initially loosely connect button 12, plate 14 and second button 16. The connection is preferably made using set of sutures 18 that initially includes four sutures 18. It is understood that second button 16 can also take the configuration of a plate 14 and first button 12 combination as described herein.

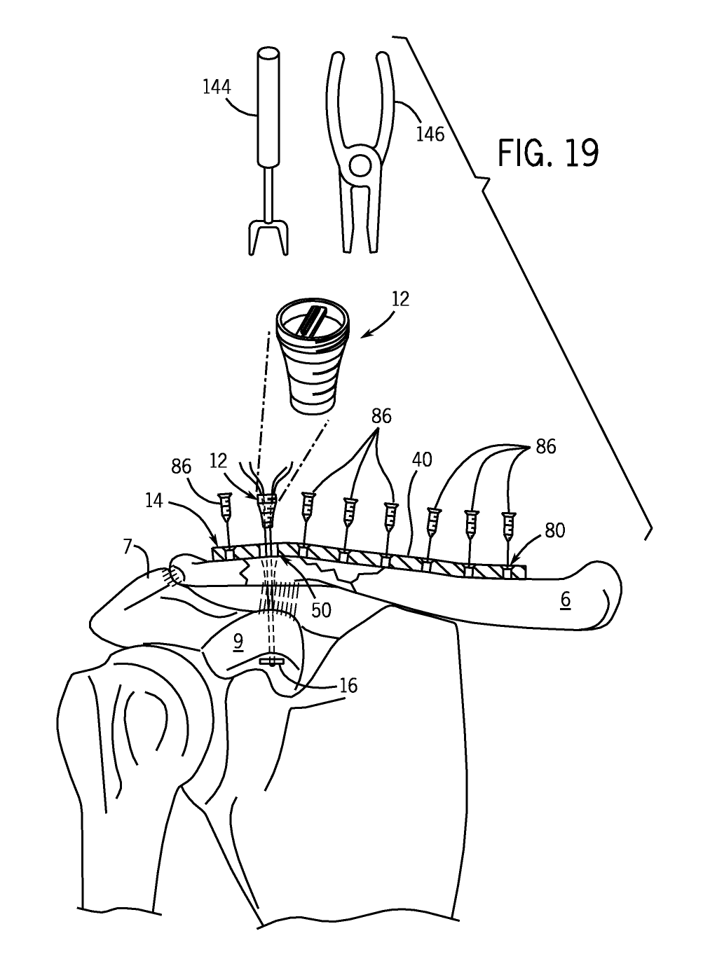

Referring now to FIGS. 2, 19 and 20, the drive mechanism is used to thread and countersink fasteners 86 into their respective apertures 80 in plate 14. The drive mechanism interfaces with notches 28 or bases 126 and 130 to rotate first button 12 into the connected position in aperture 50 of plate 14. The opposing suture 18 terminal end portions are pulled taught and fixed in position under tension using pliers and/or crimpers 146 to compress malleable walls 128 and 132 together. In addition, the terminal ends of two of the four sutures 18 of set of sutures 18 are passed through aperture 140 and then set of sutures 18 are knotted over malleable wall 132. The knotted sutures are recessed in the suture well. Any excess length of sutures 18 can be trimmed off or tucked distally into button 12. The connection of sutures 18 to first button 12 places suture button apparatus 10 in the second position that includes the connection of clavicle 6 to coracoid process 9 using second button 16 for fixation and the connection of plate 14 and first button 12 for the stabilization of clavicle 6.

Once the treatment period is completed, set of sutures 18 are released using the processes previously described. First button 12, second button 16 set of sutures 18, fasteners 86 and plate 14 can then be removed from clavicle 6 and coracoid process 9.

In the preceding specification, the present disclosure has been described with reference to specific exemplary embodiments thereof. It will be evident, however, that various modifications, combinations and changes may be made thereto without departing from the broader spirit and scope of the invention as set forth in the claims that follow. For example, it is understood that suture button apparatus 10 can be operatively employed in alternative settings such as, for example, to treat other joint related injuries such as ankle and bunion injuries. Similarly, while first button 12 and/or bar 30 are described as having threaded external interfaces, alternative external interfaces can include snap and various friction interfaces. Thus, the embodiments described herein can be applied or combined as novel features between the embodiments described herein. As a further example, bar 30 as a pair of malleable walls can also be removable and replaceable from tubular wall 20. Similarly, removable and replaceable annular bar 30 can also include a secondary fixation device as described for the malleable wall bar 30. The drawings and specification are accordingly to be regarded in an illustrative manner rather than a restrictive sense.

What is claimed is:

1. A suture button apparatus that comprises:
a first button that includes a tubular wall that has a proximal end portion and an opposed distal end portion, the tubular wall defines a central longitudinal axis, the proximal end portion of the tubular wall includes a proximal terminal end and an external interface adapted to connect to an external drive mechanism, the tubular wall has an inner surface and an outer surface, the inner surface defines an aperture aligned with the central longitudinal axis, the outer surface of the tubular wall includes threads;
a plate that includes a first side and an opposed second side connected by a sidewall, the plate defines an aperture that extends through the first side and the second side, the first side receives the first button, the aperture of the plate includes threads that mate with at least a portion of the threads of the first button;
a second suture button that includes at least one aperture;
a set of sutures that connect the first button and the second button; and
a bar positioned in the aperture of the first button and connected to the tubular wall, the bar positioned distal to the proximal terminal, the bar includes a fixation mechanism that fixes the set of sutures relative to the first button; and
a position for treatment that includes the set of sutures connecting the first button and the second button, the first button connected to the aperture of the plate and the proximal terminal end of the first button at least flush with the first side of the plate.

2. The suture button apparatus of claim 1, wherein the external interface is a pair of opposed notches defined in the tubular wall, the notches extend distally from the proximal terminal end of the tubular wall for a predetermined distance, the notches adapted to connect to the external drive mechanism.

3. The suture button apparatus of claim 2, wherein the bar is selectively connected and disconnected from the first button.

4. The suture button apparatus of claim 3, wherein the bar defines the fixation mechanism between a sidewall of the removable bar and the inner surface of the tubular wall.

5. The suture button apparatus of claim 1, wherein the bar has a first position that includes two malleable walls separated by an aperture, the set of sutures positionable through the aperture between the walls, the fixation mechanism includes moving the walls between the first position and a second position that fixes the position of the set of sutures between the walls.

6. The suture button apparatus of claim 1, wherein the first button includes a first portion that has a first set of threads that interface with the plate and a second portion that has a second set of threads.

7. The suture button apparatus of claim 5, wherein the tubular wall of the first button includes a shaft and the outer surface of the shaft has threads.

8. A method of treating a fractured bone, the method comprising the steps of:
providing a suture button apparatus that comprises a first button, a second button, a set of sutures and a plate, the first button includes a tubular wall that has a distal end and a proximal end, the first button including a bar positioned in an aperture defined in the first button and distal to a proximal end of the first button;
positioning the second button at a location that supports the fixation of the fractured bone;
positioning the first button at least partially through the plate and into an aperture connecting the first and second buttons for the fixation of the fractured bone,
connecting the plate, first button and second button together using the set of sutures;
connecting the first button into a first side of the plate such that the proximal end of the first button is at least flush with a first side of the plate; and
using a fixation mechanism of the bar to fix the position of the set of sutures relative to the first button.

9. The method of treating a fractured bone of claim 8, wherein the step of positioning the first button includes threading the first button into the fractured bone.

10. The method of treating a fractured bone of claim 8, wherein the step of using a fixation mechanism to fix the position of the set of sutures includes knotting the sutures against the bar.

11. The method of treating a fractured bone of claim 8, wherein the step of using a fixation mechanism further includes using a removable bar to secure the set of sutures between the bar and the inner surface of the tubular wall of the first button.

12. The method of treating a fractured bone of claim 8, wherein the step of using a fixation mechanism further includes the bar including a pair of malleable walls and the set of sutures extending between the walls and crimping the pair of malleable walls together fixing the position of the set of sutures between the walls.
